(19) United States
(12) Patent Application Publication
(10) Pub. No.: US 2005/0165099 A1
(43) Pub. Date: Jul. 28, 2005

(54) Method for producing 2-(4-n,n-dialkylamino-2-hydroxybenzol)benzoates

(76) Inventors: Thomas Heidenfelder, Dannstadt (DE); Karl Beck, Ostringen (DE)

Correspondence Address: NOVAK DRUCE DELUCA & QUIGG, LLP, 1300 EYE STREET NW, SUITE 400 EAST, WASHINGTON, DC, 20005, US

(21) Appl. No.: 10/514,410

(22) PCT Filed: May 12, 2003
(86) PCT No.: PCT/EP03/04919

(30) Foreign Application Priority Data
May 15, 2002 (DE) 102 21 805.6

(51) Int. Cl. A61K 31/24; C07C 229/52

(52) U.S. Cl. 514/539; 560/45

(57) ABSTRACT

The invention relates to a method for producing 2-(4-N,N-dialkylamino-2-hydroxybenzoyl)benzoates of formula (I), in which the substituents R¹ to R³, independently of one another, are defined as cited in the description. Said substances are produced as follows: L 3-N,N-dialkylamimophenol of formula (II) is reacted with phthalic anhydride of formula (III) to obtain 2-(4-N,N-dialkylamino-2-hydroxybenzoyl) benzoic acid of formula (IV) and II. said 2(4-N,N-dialkylamino-2-hydroxybenzoyl) benzoic acid of formula (IV) that has been formed in stage I. is esterified by means of a C₁-C₁₂ alcohol or a cyclic C₃-C₁₀ alcohol in the presence of an acidic catalyst to obtain 2-(4-N,N-dialkylamino-2-hydroxybenzoyl)benzoate of formula (I). The method is characterised in that the ester of formula (I) that has been formed is purified in an additional stage m by treatment with an adsorbent and/or by distillation.

The invention relates to a process for the preparation of 2-(4-N,N-dialkylamino-2-hydroxybenzoyl)benzoic esters.

Sunlight which reaches the earth's surface has a content of UV-B radiation (280 to 320 nm) and of UV-A radiation (>320 nm), which border directly the visible light region. The effect on human skin is particularly evident in the case of UV-B radiation from sunburn. Accordingly, the industry offers a relatively large number of substances which absorb UV-B radiation and thus prevent sunburn.

Dermatological investigations have shown that UV-A radiation is also entirely capable of causing skin damage and allergies by, for example, harming the keratin or elastin. This reduces elasticity and water storage capacity of the skin, i.e. the skin becomes less supple and tends toward wrinkling. The markedly high incidence of skin cancer in regions of strong solar irradiation shows that damage to the genetic information in the cells is apparently also caused by sunlight, specifically by UV-A radiation. All of these findings therefore suggest the need to develop efficient filter substances for the UV-A and UV-B region.

Substances which have a benzophenone structure 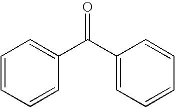
are characterized by very good absorption properties in the UV-A region. Representatives of this class of substance are inter alia 2-(4-N,N-dialkylamino-2-hydroxybenzoyl)benzoic alkyl esters. Their use as photostable UV filters in cosmetic or pharmaceutical preparations is described in DE-A-199 17 906.

According to DE-A-199 17 906, the abovementioned 2-(4-N,N-dialkylamino-2-hydroxybenzoyl)benzoic alkyl esters can be prepared by direct acylation of the corresponding amino-substituted phenols with phthalic anhydride to give keto acids and subsequent esterification.

As is known, the reaction of amino-substituted phenols with phthalic anhydride produces rhodamines as by-products, which lead to an undesired discoloration of the keto acids formed.

To avoid/reduce the rhodamine formation, EP-B-0 511 019 describes a process for the preparation of a keto acid of the formula 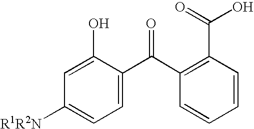
where R¹ and R², independently, are alkyl having 1-6 carbon atoms or cycloalkyl having 4-8 carbon atoms, comprising the reaction of an m-aminophenol of the formula 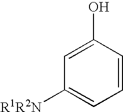
with phthalic anhydride in the presence of an organic solvent, where the organic solvent is present in an amount of from 0.5 to 3 parts by weight per 1 part by weight of the m-aminophenol, with the effect that the resulting keto acid is precipitated in the solvent, meaning that the reaction is carried out in a slurry.

EP-A-0 853 079 likewise includes a process for the preparation of the abovementioned keto acids by reaction of an m-aminophenol with phthalic acid in the presence of an organic solvent, where the organic solvent is present in an amount of less than 0.5 part by weight per 1 part by weight of the m-aminophenol.

The 2-(4-N,N-dialkylamino-2-hydroxybenzoyl)benzoic alkyl esters prepared in accordance with the abovementioned processes also often exhibit an undesired discoloration and thus do not satisfy the high quality standards which are required of these compounds for use as UV filters in cosmetic preparations.

It is an object of the present invention to provide a process for the preparation of 2-(4-N,N-dialkylamino-2-hydroxybenzoyl)benzoic esters which can be carried out easily and leads to a colorless product with high purity.

We have found that this object is achieved by a process for the preparation of 2-(4-N,N-dialkylamino-2-hydroxybenzoyl)benzoic esters of the formula I, 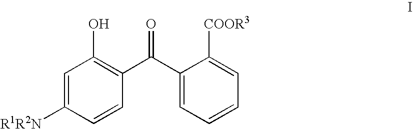
in which the substituents, independently of one another, have the following meanings:
R¹ and R²
   are C₁-C₆-alkyl, C₃-C₁₀-cycloalkyl;
R³ is C₁-C₁₂-alkyl, C₃-C₁₀-cycloalkyl
by
I. reaction of 3-N,N-dialkylaminophenol of the formula II, in which R¹ and R² have the meanings given above, with phthalic anhydride of the formula III to give 2-(4-N,N-dialkylamino-2-hydroxybenzoyl)benzoic acid of the formula IV and 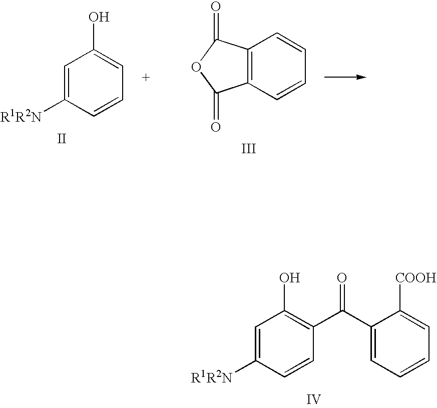
II. subsequent esterification of the 2-(4-N,N-dialkylamino-2-hydroxybenzoyl)benzoic acid of the formula IV formed in stage I with a C₁-C₁₂-alcohol or a cyclic C₃-C₁₀-alcohol in the presence of an acidic catalyst to give the C₁-C₁₂-alkyl 2-(4-N,N-dialkylamino-2-hydroxybenzoyl)benzoic esters of the formula I, 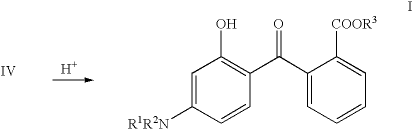
   which comprises purifying the ester of the formula I formed in a further process stage III by treatment with an adsorbent and/or by distillation.

Alkyl radicals for R¹ and R² which may be mentioned are branched or unbranched C₁-C₆-alkyl chains, such as methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl and 1-ethyl-1-methylpropyl.

Particularly preferred alkyl radicals for R¹ and R² are the C₁-C₄-alkyl chains mentioned in the above group, very particularly preferably the C₁-C₃-alkyl chains, such as methyl, ethyl, n-propyl and 1-methylethyl.

Cycloalkyl radicals which may be mentioned for R¹ to R³ are preferably branched or unbranched C₃-C₁₀-cycloalkyl chains, such as cyclopropyl, cyclobutyl, cyclopentyl, cyclohexyl, cycloheptyl, 1-methylcyclopropyl, 1-ethylcyclopropyl, 1-propylcyclopropyl, 1-butylcyclopropyl, 1-pentylcyclopropyl, 1-methyl-1-butylcyclopropyl, 1,2-dimethylcyclopropyl, 1-methyl-2-ethylcyclopropyl, cyclooctyl, cyclononyl or cyclodecyl.

The cycloalkyl radicals may optionally be substituted by one or more, e.g. 1 to 3, radicals, such as halogen, e.g. fluorine, chlorine or bromine, cyano, nitro, amino, C₁-C₄-alkylamino, C₁-C₄-dialkylamino, hydroxy, C₁-C₄-alkyl, C₁-C₄-alkoxy or other radicals or contain 1 to 3 heteroatoms, such as sulfur, nitrogen, whose free valences may be saturated by hydrogen or C₁-C₄-alkyl, or contain oxygen in the ring.

Alkyl radicals for R³ which may be mentioned are branched or unbranched C₁-C₁₂-alkyl chains, such as methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, 2-ethylhexyl, n-octyl, n-nonyl, n-decyl, n-undecyl and n-dodecyl.

Particularly preferred alkyl radicals for R³ are the C₃-C₈-alkyl chains mentioned in the above group, very particularly preferably the C₄-C₈-alkyl chains, such as n-butyl, 1,1-dimethylethyl, n-pentyl, n-hexyl, n-heptyl, 2-ethylhexyl and n-octyl.

The acylation in the process step I. is preferably carried out in the presence of a solvent. The solvents used here are, for example, aromatic hydrocarbons, such as benzene, toluene or xylene, aliphatic C₈-C₁₂-hydrocarbons, such as octane, isooctane or decane, ethers, such as diethyl ether, dibutyl ether or tetrahydrofuran, and chlorinated hydrocarbons, such as perchloroethylene or chlorobenzene. Particularly preferred solvents are toluene and xylene.

The amount of solvent used is generally chosen such that the keto acid formed crystallizes out during the reaction. Depending on the chain length of the substituents R¹ and R², the organic solvent can be used in an amount of from 0.5 to 5 parts by weight, preferably from 1 to 4.5 parts by weight, particularly preferably from 3.5 to 4.5 parts by weight per 1 part by weight of the 3-N,N-dialkylaminophenol. For reasons of stirrability of the reaction mixture, for sparingly soluble keto acids the amount of solvent should be in the range from 3.5 to 4.5 parts by weight per 1 part by weight of the 3-N,N-dialkylaminophenol.

It is also possible to use for the acylation a solvent in amounts in the range greater than 3.5 parts by weight per 1 part by weight of the 3-N,N-dialkylaminophenol, it having proven advantageous if in this case some of the solvent is in turn distilled off during the reaction.

The reaction temperature at which the acylation is carried out is generally in the range between 50° C. and 150° C., preferably the boiling temperature of the solvent used.

The molar ratio of the reactants, phthalic anhydride to 3-N,N-dialkylaminophenol, is generally in the range from 0.7:1 to 2:1, preferably in the range from 1:1 to 1.5:1.

When the reaction is complete, the keto acid [2-(4-N,N-dialkylamino-2-hydroxybenzoyl)benzoic acid] formed can, after the reaction mixture has been cooled to temperatures in the range between 0° C. and 60° C., preferably between 10° C. and 50° C., particularly preferably between 30° C. and 50° C., be filtered off, washed with the solvent and then used directly and without drying in the second stage (esterification).

The esterification of the 2-(4-N,N-dialkylamino-2-hydroxybenzoyl)benzoic acid IV formed in stage I is carried out in a manner known per se (see for this Organikum, VEB Deutscher Verlag der Wissenschaften, Berlin 1986, 16th Edition, pages 400-408) with the corresponding C₁-C₁₂-alcohol or cyclic C₃-C₁₀-alcohol in the presence of an acidic catalyst. The alcohol used can function here both as a reagent and as a solvent. To increase the yield, it is advantageous if the water of reaction formed during the esterification is removed by azeotropic distillation.

Acid catalysts which may be used are, for example, HCl, H₂SO₄, HNO_{3,} phosphoric acid, sulfonic acids, such as benzenesulfonic acid, p-toluenesulfonic acid, methanesulfonic acid or mixtures of these acids, but also sulfonic acid group-containing ion exchangers, such as, for example, Lewatits S100 (Bayer). Preferred acidic catalysts are HCl, H₂SO₄, methanesulfonic acid and p-toluenesulfonic acid.

A particularly preferred embodiment of the process according to the invention involves the esterification in the process stage II being carried out in the presence of sulfuric acid as catalyst.

When the esterification is complete, the reaction mixture is neutralized and the ester is isolated after separating off the aqueous phase.

The adsorbents used in the process step III are generally solid substances which, due to their large surface area, are able to selectively adsorb impurities from liquid mixtures at their interface. Preference is given to adsorbents chosen from the group consisting of activated carbons, aluminum oxides, zeolites and silica gels. Particularly preferred adsorbents are activated carbons and silica gels.

Of the aluminum oxides, basic, neutral or else acidic aluminum oxides may be used. Advantageously, the “active” aluminum oxides, which are obtained, for example, via thermally after-treated aluminum hydroxide gels or by calcination from α-aluminum hydroxide, are used.

Of the zeolites, the synthetic zeolites are of particular interest as adsorbent. Details on the composition and structure of these zeolites are given in the CD Römpp Chemie Lexikon—Version 1.0, keyword: zeolites, Stuttgart/New York: Georg Thieme Verlag 1995 and the literature cited therein.

The silica gels suitable as adsorbents are described, inter alia, in the CD Rompp Chemie Lexikon—Version 1.0, keyword: silica gels, Stuttgart/New York: Georg Thieme Verlag 1995 and the literature cited therein. Preferred silica gels are silica gel 60 from Merck, Darmstadt and silica gel 123 from Grace.

A preferred embodiment of the process is the purification of the ester of the formula I formed by treatment with activated carbon. Here, the activated carbon may be used in powder form, granule form or as cylindrically formed particles. In this connection, the activated carbon is advantageously used in granule form (granular activated carbon) in fixed- or fluidized-bed filters. Examples of preferred carbons are the activated carbons CPG® LF, CAL® and APC® from Chemviron Carbon. Further details on properties and grades of the activated carbons used are given in Ullmann's Encyclopedia, Sixth Edition, 2000 Electronic Release, Chapter 5.

In the case of the aluminum oxides, zeolites and silica gels, it is likewise advantageous if these adsorbents are used as a fixed bed.

The amount of adsorbent used is in the range from 0.001 to 0.2 g, preferably 0.05 to 0.1 g, based in each case on 1 g of the ester I to be purified.

The process according to the invention also comprises crystallizing the 2-(4-N,N-dialkylamino-2-hydroxybenzoyl)benzoic ester of the formula I from the alcoholic solution prior to the treatment with an adsorbent and/or distillation.

The process according to the invention is further distinguished by the fact that the 2-(4-N,N-dialkylamino-2-hydroxy]benzoyl)benzoic ester of the formula I formed comprises less than 10 ppm, preferably less than 5 ppm, particularly preferably less than 1 ppm, of rhodamine.

A particularly preferred embodiment of the process using activated carbon comprises, in process stage III,
a. dissolving the ester in a nonpolar solvent at a temperature in the range from 10° C. to 100° C., preferably in the range from 20° C. to 80° C., particularly preferably in the range from 25° C. to 50° C.,
b. passing this solution over a granular activated carbon bed at a temperature in the range from 10° C. to 100° C., preferably in the range from 20° C. to 80° C., particularly preferably in the range from 25° C. to 50° C.,
c. after it is passed through the granular activated carbon bed, separating off the ester from the solvent by distillation.

It is also possible, in the process stage III,
a. to dissolve the ester in a nonpolar solvent at a temperature in the range from 10° C. to 100° C., preferably in the range from 20° C. to 80° C., particularly preferably in the range from 25° C. to 50° C.,
b. to suspend in this solution pulverulent adsorbent, in particular pulverulent activated carbon, at a temperature in the range from 20° C. to 100° C., preferably in the range from 40° C. to 80° C., and to stir the suspension for 0.1 to 6 hours, preferably 1 to 3 hours,,
c. to filter off the adsorbent, in particular the activated carbon, and
d. to crystallize the ester out of the solution separated off from the activated carbon at a temperature in the range from 0° C. to 40° C., preferably in the range from 5° C. to 20° C., to filter it and then it to dry it.

For the purposes of the invention, nonpolar solvents means those solvents with low dielectric constants (ε<15) and small dipole moment (μ=0 to 2). Examples are, inter alia, petroleum ether, ligroin, n-hexane, cyclohexane, heptane, di-n-butyl ether, xylene, toluene and benzene. A preferred solvent used in process step IIIa is toluene, hexane or cyclohexane, particularly preferably toluene or cyclohexane, very particularly preferably toluene.

It is also possible, in the process stage III,
a. to dissolve the ester in an alcohol, in particular in hexanol, at a temperature in the range 10° C. to 100° C., preferably in the range from 20° C. to 80° C., particularly preferably in the range from 25° C. to 50° C.,
b. to pass this solution over a silica gel bed at a temperature in the range from 10° C. to 100° C., preferably in the range from 20° C. to 80° C., particularly preferably in the range from 25° C. to 50° C.,
c. after it has passed through the silica gel bed, to separate off the ester from the alcohol by distillation.

A preferred embodiment of the above process according to the invention is one where the benzoic alkyl ester is n-hexyl 2-(4-N,N-diethylamino-2-hydroxybenzoyl)benzoate of the formula Ia.

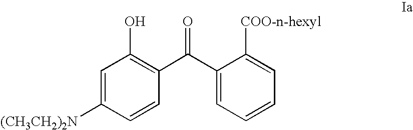

Advantageously, this ester is purified by treatment with an adsorbent, in particular with activated carbon, and subsequent distillation.

A very particularly preferred embodiment of the process relates to the preparation of n-hexyl 2-(4-N,N-diethylamino-2-hydroxybenzoyl)benzoate of the formula Ia 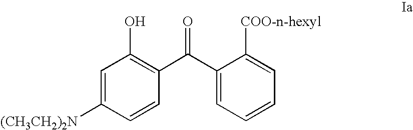
by
I. reaction of 3-N,N-diethylaminophenol of the formula IIa with phthalic anhydride of the formula III to give 2-(4-N,N-diethylamino-2-hydroxybenzoyl)benzoic acid of the formula IVa, 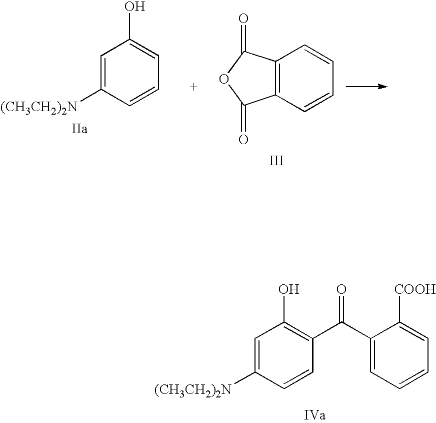
II. esterification of the 2-(4-N,N-diethylamino-2-hydroxybenzoyl)benzoic acid of the formula IVa formed in stage I in hexanol in the presence of sulfuric acid to give n-hexyl 2-(4-N,N-diethylamino-2-hydroxybenzoyl)benzoate of the formula Ia 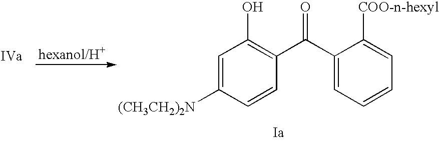
   and isolation of the n-hexyl ester Ia in crystalline form,
III.
a. dissolution of the n-hexyl ester Ia in toluene or hexanol at a temperature in the range from 25° C. to 50° C.,
b. metering of this solution over a granular activated carbon bed or a silica gel bed at a temperature in the range from 25° C. to 50° C. and
c. isolation of the n-hexyl ester by separating off the toluene and/or hexanol by distillation.

The distillative purification is generally carried out by firstly separating off the solvent, for example via a falling-film or thin-layer evaporator under reduced pressure, and then distilling the residue containing the product of value over a column.

Following the distillation, the colorless ester obtained in this way can preferably be packaged as a melt.

The examples below serve to illustrate the process according to the invention in more detail.

### EXAMPLE 1

Preparation of 2-(4-N,N-diethylamino-2-hydroxybenzoyl)benzoic acid

99 g (0.60 mol) of 3-diethylaminophenol, 93.2 g (0.63 mol) of phthalic anhydride and 460 ml of toluene were introduced, under a nitrogen atmosphere, into a 500 ml four-necked flask fitted with Teflon stirrer, thermometer and reflux condenser, and heated to the reflux temperature. After a reaction time of 2 hours, a total of 300 g of toluene were distilled off over the course of 30 minutes, and then the mixture was stirred under reflux for 3 hours. The mixture was cooled to room temperature and filtered with suction. The filter cake was washed successively with 90 ml of toluene and with 2×90 ml of hexanol. The hexanol-moist acid could be used directly in the second stage. Yield: 169 g (90%) of pink-colored 2-(diethylamino-2-hydroxybenzoyl)benzoic acid.

### EXAMPLE 2

Preparation of n-hexyl 2-(4-N,N-diethylamino-2-hydroxybenzoyl)]benzoate

313 g (1.0 mol) of hexanol-moist 2-(4-diethylamino-2-hydroxybenzoyl)benzoic acid (calc. 100%) and 750 ml of n-hexanol were introduced into a 1 l flat-flange flask fitted with anchor stirrer, nitrogen inlet, thermocouple and water separator, admixed with 53 g (519 mmol) of 96% strength sulfuric acid and heated to an internal temperature of 105-110° C. After the heating phase, the water of reaction formed was distilled off azeotropically over the course of 6-8 h at an internal temperature of 105-110° C. and a pressure of about 200 mbar. After cooling to about 70° C., the mixture was admixed with 830 ml of water and, at a temperature of 52-58° C., neutralized with 25% strength NaOH solution. The aqueous phase was separated off and the organic phase was extracted with 500 ml of water (temperature: 52-58° C.). The aqueous phase was separated off, the organic phase was cooled to 20° C. and crystallization of the ester awaited. Following a holding phase of 1 hour for maturation of the in-situ generated seed material, the mixture was cooled to 0 to 5° C. at 5 K/h, after-stirred for 2 h at this temperature and filtered with suction. The filtercake was washed with 2×85 ml of cold hexanol. The hexanol-moist, pink-colored crude product (407 g) was thoroughly dried with suction and purified by adsorption on activated carbon.

### EXAMPLE 3

Purification of n-hexyl 2-(4-N,N-diethylamino-2-hydroxy]benzoyl)benzoate using granular activated carbon

200 g of moist n-hexyl 2-(4-N,N-diethylamino-2-hydroxybenzoyl)]benzoate, obtainable as in Example 2, were dissolved in 400 ml of toluene and passed, at 25° C., over a column filled with activated carbon CPG® LF from Chemviron Carbon. The colorless eluate was concentrated by means of a falling-film evaporator and the oily residue was depleted to <10 ppm of toluene in a distillation column at a pressure of 100 mbar in countercurrent with hot nitrogen. The product of value was then drawn off as a melt.

### EXAMPLE 4

Purificatin of n-hexyl 2-(4-N,N-diethylamino-2-hydroxy]benzoyl)benzoate using pulverulent activated carbon

75 g of n-hexyl 2-(4-N,N-diethylamino-2-hydroxybenzoyl)benzoate and 4 g of Carbopal® PC 250 (Donau Carbon) were admixed with 190 ml of cyclohexane and stirred for 2 h at 60° C. The activated carbon was then filtered off hot and the filtrate, for crystallization of the ester, was cooled to 10° C. and stirred for 1 h. The crystallized-out ester was filtered off, washed with cold cyclohexane and then dried at 35° C./200 bar.

### EXAMPLE 5

Purification of n-hexyl 2-(4-N,N-diethylamino-2-hydroxy]benzoyl)benzoate using calcined aluminum oxide

200 g of moist n-hexyl 2-(4-N,N-diethylamino-2-hydroxybenzoyl))benzoate, obtainable as in Example 2, were dissolved in 400 ml of hexanol and, at 25° C., passed over a column filled with calcined aluminum oxide (Calcined Aluminas® from Alcoa Inc.). The colorless eluate was concentrated using a falling-film evaporator, and the oily residue was depleted to <10 ppm of toluene in a distillation column at a pressure of 100 mbar in countercurrent with hot nitrogen. The product of value was then drawn off as a melt.

1. A process for the preparation of 2-(4-N,N-dialkylamino-2-hydroxybenzoylibenzoic esters of the formula I, 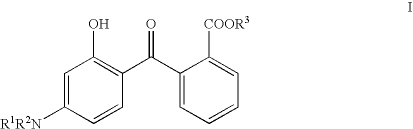
in which the substituents, independently of one another, have the following meanings:
R¹ and R²
are C₁-C₆-alkyl, C₃-C₁₀-cycloalkyl selected from the group consisting of cyclopropyl, cyclobutyl, cyclopentyl, cyclohexyl, cycloheptyl, 1-methylcyclopropyl, 1-ethylcyclopropyl, 1-propylcyclopropyl, 1-butylcyclopropyl, 1-pentacyclopropyl, 1-methyl-1-butylcyclopropyl, 1,2-dimethylcyclypropyl, 1-methyl-2-ethylcyclopropyl, cyclooctyl, cyclooctyl and cyclodecyl;
R³ is C₁-C₁₂-alkyl, C₃-C₁₀-cycloalkyl selected from the group consisting of cyclopropyl, cyclobutyl, cyclopentyl, cyclohexyl, cycloheptyl, 1-methylcyclopropyl, 1-ethylcyclopropyl, 1-propylcyclopropyl, 1-butylcyclopropyl, 1-pentacyclopropyl, 1-methyl-1-butylcyclopropyl, 1,2-dimethylcyclypropyl, 1-methyl-2-ethylcyclopropyl, cyclooctyl, cyclooctyl and cyclodecyl by
I. reaction of 3-N,N-dialkylaminophenol of the formula II, in which R¹ and R² have the meanings given above” with phthalic anhydride of the formula III to give 2-(4-N,N-dialkylamino-2-hydroxybenzoyl)benzoic acid of the formula IV and 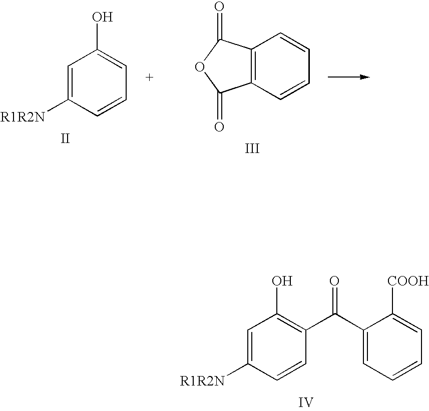
II. subsequent esterification of the 2-(4-N,N-dialkylamino-2-hydroxybenzoyl)benzoic acid of the formula IV formed in stage I with a C₁-C₁₂-alcohol or a cyclic C₃-C₁₀-alcohol in the presence of an acidic catalyst to give the 2-(4-N,N-dialkylamino-2-hydroxybenzoyl)benzoic ester of the formula I, 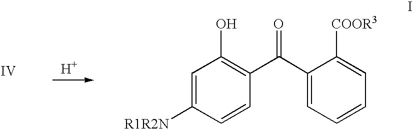
which comprises crystallizing the ester of the formula I formed and purifying the crystals in a further process stage III by treatment with an adsorbent and/or by distillation.

2. A process as claimed in claim 1, wherein the adsorbent is a substance chosen from the group consisting of activated carbons, aluminum oxides, zeolites and silica gels.

3. A process as claimed in claim 1, wherein the esterification in the process stage II is carried out in the presence of sulfuric acid as catalyst.

4. A process as claimed in any of claims 1 to 3, wherein the 2-(4-N,N-dialkylamino-2-hydroxybenzoyl)benzoic ester of the formula I formed comprises less than 10 ppm of rhodamine.

5. A process as claimed in any of claims 1 to 4, wherein the benzoic ester is n-hexyl 2-(4-N,N-diethylamino-2-hydroxybenzoyl)benzoate of the formula Ia

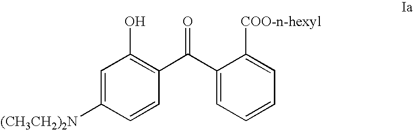

6. A process as claimed in claim 1, wherein, in the process stage III, the adsorbent used is activated carbon or silica gel.

7. A process as claimed in claim 6, wherein, in process stage III, the ester is purified by treatment with activated carbon and subsequent distillation.

8. A process as claimed in claim 7, wherein, in the process stage III
a. the ester is dissolved in a nonpolar solvent at a temperature in the range from 10° C. to 100° C.,
b. this solution is passed over a granular activated carbon bed at a temperature in the range from 20° C. to 100° C.,
c. the ester, after passing through the granular activated carbon bed, is separated off from the solvent by distillation.

9. A process as claimed in claim 8, wherein the solvent used in the process step IIIa is cyclohexane or toluene.

10. A process for the preparation of n-hexylyl 2-(4-N,N-diethylamino-2-hydroxybenzoyl)benzoate of the formula Ia 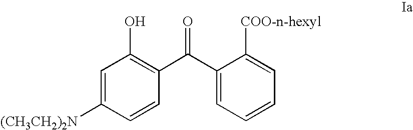
by
I. reaction of 3-N,N-diethylaminophenol of the formula IIa with phthalic anhydride of the formula III to give 2-(4-N,N-diethylamino-2-hydroxybenzoyl)benzoic acid of the formula IVa, 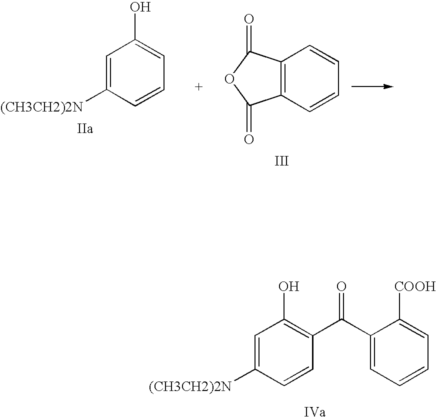
II. esterification of the 2-(4-N,N-diethylamino-2-hydroxybenzoyl)benzoic acid of the formula IVa formed in stage I in hexanol in the presence of sulfuric acid to give n-hexyl 2-(4-N,N-diethylamino-2-hydroxylbenzoyl)benzoate of the formula Ia 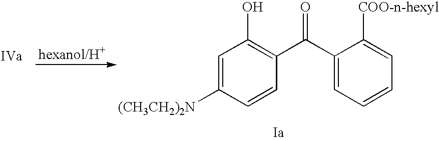
and isolation of the n-hexyl ester Ia in crystalline form,
III.
a. dissolution of the n-hexyl ester Ia in toluene or hexanol at a temperature in the range from 25° C. to 50° C.,
b. metering of this solution over a granular activated carbon bed or a silica gel bed at a temperature in the range from 25° C. to 50° C. and
c. subsequent isolation of the n-hexyl ester by separating off the toluene and/or hexanol by distillation.
